(19) United States
(12) Patent Application Publication
(10) Pub. No.: US 2022/0330964 A1
(43) Pub. Date: Oct. 20, 2022

(54) LOCKING FORCEPS DEVICE

(71) Applicant: ECA Medical Instruments, Inc., Thousand Oaks, CA (US)

(72) Inventor: Randall Allard, Thousand Oaks, CA (US)

(21) Appl. No.: 17/707,844

(22) Filed: Mar. 29, 2022

Related U.S. Application Data
(60) Provisional application No. 63/167,591, filed on Mar. 29, 2021.

(51) Int. Cl. A61B 17/28

(52) U.S. Cl. CPC A61B 17/2833; A61B 2017/2837

(57) ABSTRACT

Disclosed herein are forceps devices, for example, forceps devices for use in medical procedures such as those involving bone reduction. These forceps devices can comprise features that allow for locking or restricting the motion of the device, for example engagement features and corresponding and complementary opposing features. In some embodiments, the engagement features comprise teeth and the opposing features comprise indentations. In some embodiments, the engagement features comprise teeth, and the opposing features comprise teeth comprising a complementary opposing angle to the angle of the engagement features.

This application claims the benefit of U.S. Provisional Patent Application Ser. No. 63/167,591, filed on Mar. 29, 2021. The application referred to in this paragraph is incorporated by reference as if set forth fully herein.

### BACKGROUND

### Related Field

The present disclosure is related generally to instruments for use in the medical field for surgical procedures, and specifically, forceps devices for surgical applications.

### Description of the Related Art

The use of surgical instruments, including forceps devices, is common in the medical industry. Surgical hand tools, such as ratcheting screwdrivers, are used for a variety of reasons in surgical settings.

However, conventional forceps designs can be improved to provide better functionality during surgery, for example, when utilized in the case of tenaculums for use in procedures involving bone reduction. Furthermore, conventional forceps devices have designs that can only function properly utilizing the conventional metal-based materials currently in use. As the focus shifts to utilizing single-use disposable medical tools, for example, sold and packaged together as “kits,” forceps designs that can support tools comprising different materials are desired.

### SUMMARY

Embodiments incorporating features of the present disclosure include forceps devices configured to have locked and moveable positions. Forceps devices incorporating features of the present disclosure can comprise features, such as teeth, configured to interact with complementary features, such as indentations, opposing teeth, or the like which can prevent further operation of the forceps when engaged. For example, a surgeon can cause the engagement features and opposing features to engage when the surgeon wants to retain the forceps in a clamped position and can disengage the engagement features and opposing features when the surgeon wants to open the clamped forceps.

These and other further features and advantages of the disclosure would be apparent to those skilled in the art from the following detailed description, taken together with the accompanying drawings, wherein like numerals designate corresponding parts in the figures, in which:

### BRIEF DESCRIPTION OF THE DRAWINGS

FIG. 1 shows a top perspective view of an embodiment of a forceps device incorporating features of the present disclosure;

FIG. 2 shows a front perspective view of the embodiment of FIG. 1;

FIG. 3 shows a zoomed-in front perspective operational view of the engagement portion of the embodiment of FIG. 1, shown in a first operational state wherein the engagement features and opposing features are shown in a disengaged state;

FIG. 4 shows a zoomed-in front perspective operational view of the engagement portion of the embodiment of FIG. 1, shown in a second operational state wherein the engagement features and opposing features are shown in an engaged state;

FIG. 5 shows a top perspective view of another embodiment of a forceps device incorporating features of the present disclosure;

FIG. 6 shows a bottom perspective view of the embodiment of FIG. 5;

FIG. 7 shows a close-up perspective operational view of the engagement portion of the embodiment of FIG. 5, shown in a first operational state wherein the engagement features and opposing features are shown in a disengaged state; and

FIG. 8 shows a close-up perspective operational view of the engagement portion of the embodiment of FIG. 5, shown in a second operational state wherein the engagement features and opposing features are shown in an engaged state.

FIG. 9 shows a perspective view of the embodiment of FIG. 1.

FIGS. 10a and 10b show close-up perspective views of the pivot point of the embodiment of FIG. 1.

### DETAILED DESCRIPTION

In the following detailed description, numerous specific details are set forth in order to provide a more thorough understanding of embodiments incorporating features of the present disclosure. However, it will be apparent to one skilled in the art that devices and methods according to the present disclosure can be practiced without necessarily being limited to these specifically recited details.

Devices incorporating features of the present disclosure include a functional forceps device comprising engagement features that are configured to engage with opposing features such that when the engagement features and opposing features are in an engaged state, operational movement of the forceps device is locked in a certain position or is otherwise limited to an acceptably minimal range of motion. The operator of the forceps device can move the engagement features and opposing features into a disengaged state, allowing the forceps to move through an entire range of motion.

Several exemplary embodiments utilizing various engagement features and opposing features are presented herein. For example, in some embodiments, the engagement features comprise teeth and the opposing features comprise indentations. In some other embodiments, the engagement features comprise teeth and the opposing features comprise opposing teeth configured in an opposing complementary angle to the engagement features.

Throughout this description, the preferred embodiments and examples illustrated should be considered as exemplars, rather than as limitations on the present disclosure. As used herein, the term “disclosure,” “device,” “present disclosure,” or “present device” refers to any one of the embodiments of the disclosure described herein, and any equivalents. Furthermore, reference to various feature(s) of the “disclosure,” “device,” “present disclosure,” or “present device” throughout this document does not mean that all claimed embodiments or methods must include the referenced feature(s).

Furthermore, any element in a claim that does not explicitly state “means for” performing a specified function, or “step for” performing a specific function, is not to be interpreted as a “means” or “step” clause as specified in 35 U.S.C. § 112, for example, in 35 U.S.C. § 112(f) or pre-AIA 35 U.S.C. § 112, sixth paragraph. In particular, the use of “step of” or “act of” in the claims herein is not intended to invoke the provisions of 35 U.S.C. § 112.

It is also understood that when an element or feature is referred to as being “on” or “adjacent” to another element or feature, it can be directly on or adjacent the other element or feature or intervening elements or features may also be present. It is also understood that when an element is referred to as being “attached,” “connected” or “coupled” to another element, it can be directly attached, connected or coupled to the other element or intervening elements may be present. In contrast, when an element is referred to as being “directly attached,” “directly connected” or “directly coupled” to another element, there are no intervening elements present.

Please note if used relative terms such as “left,” “right,” “front,” “back,” “top,” “bottom” “forward,” “reverse,” “clockwise,” “counter-clockwise,” “outer,” “inner,” “above,” “upper,” “lower,” “below,” “horizontal,” “vertical,” and similar terms, have been used for convenience purposes only and are not intended to imply any particular fixed direction. Instead, they are used to reflect relative locations and/or directions between various portions of an object.

Although ordinal terms, e.g., first, second, third, etc., may be used herein to describe various elements or components, these elements or components should not be limited by these terms. These terms are only used to distinguish one element or component from another element or component. Thus, a first element or component discussed below could be termed a second element or component without departing from the teachings of the present disclosure.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to different views and illustrations that are schematic illustrations of idealized embodiments of the disclosure. As such, variations from the shapes of the illustrations as a result, for example, of manufacturing techniques and/or tolerances are expected. Embodiments of the disclosure should not be construed as limited to the particular shapes of the regions illustrated herein, but are to include deviations in shapes that result, for example, from manufacturing.

It is understood that when a first element is referred to as being “between” or “interposed between” two or more other elements, the first element can be directly between the two or more other elements or intervening elements may also be present between the two or more other elements. For example, if a first element is “between” or “interposed between” a second and third element, the first element can be directly between the second and third elements with no intervening elements, or the first element can be adjacent to one or more additional elements with the first element and these additional elements all between the second and third elements.

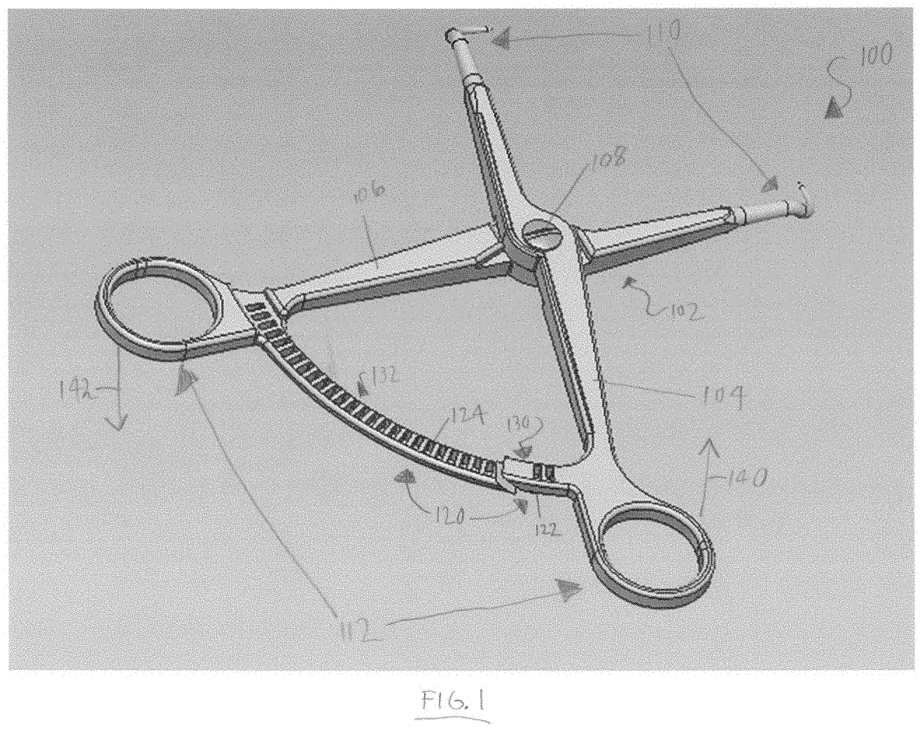

FIG. 1 shows a forceps device 100, comprising a body 102, which itself comprises a first arm 104, which is connected to a second arm 106 at a pivot point 108, which enables the first arm 104 to pivot about the pivot point 108 in relation to the second arm 106. Each of the arms 104, 106 comprises a functional end 110 and an operational end 112. The functional ends 110 can comprise the desired tip configuration to perform a particular surgical function, for example, a clamping portion as shown. The operational ends 112 can be manipulated by the user to cause the first and second arms 104, 106 to pivot about the pivot point 108, moving the functional ends 110 through a range of motion between fully open and closed operational states. For example, in one embodiment the forceps 100 may be operated by moving the operational ends 112, which in turn will move the clamp-based functional ends 110 between an open or closed position wherein the functional ends 110 are releasing or clamping an object, respectively.

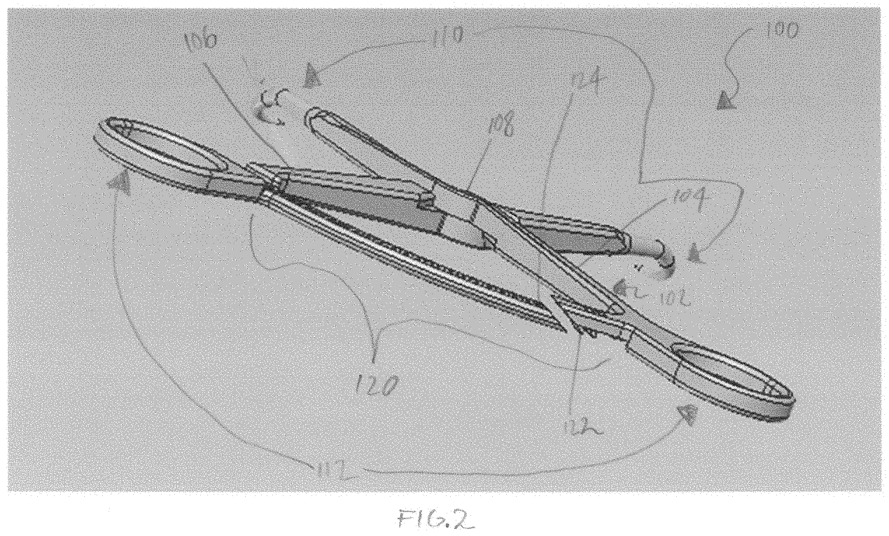

Also shown in FIG. 1 is that the forceps device 100 further comprises an engagement portion 120, comprising engagement features 122 (also shown in FIG. 2) connected to one arm, in this instance, the first arm 104 and further comprising opposing and complementary opposing features 124, configured to engage with the engagement features 122. In this embodiment, the engagement features 122 comprise protruding teeth and the opposing features 124 comprise indentations. In some embodiments, such as the embodiment shown in FIG. 1, the first arm 104 comprises a first extending component 130, and the engagement features 122 are protruding teeth formed in the first extending component 130. In this embodiment, the second arm 106 comprises a second extending component 132 comprising opposing features 124 which, in this embodiment, are indentations in the form of angled holes formed within the second extending component 132. These holes can pass all the way through the extending component 132, or they can pass through only a portion of the extending component 132 so long as there is sufficient space for the engagement features 122 to fully engage with the opposing features 124.

The forceps device 100 is configured such that movement of the operational end 112 of the first arm 104 toward the operational end 112 of the second arm 106 causes the functional ends 110 to approach one another and clamp down together. Movement of the operational end 112 of the first arm 104 toward the operational end 112 of the second arm 106 further causes the engagement features 122 to engage with the opposing features 124 preventing the operational ends from moving away from one another, causing the forceps device 100 to remain in a clamped position. The user can release the engagement features 122 from the opposing features 124, by moving the operational ends 112 of the first and second arms 104, 106 in opposing lateral directions 140, 142 (i.e., in a direction orthogonal to the plane in which the arms 104, 106 move during operation), as shown. This allows the operational ends 112 to move away from each other, which opens the functional ends 110 and releases them from a clamped state.

The body 102 can comprise a variety of materials including, but not limited to: plastics, metals, Polytetrafluoroethylene (PTFE), often marketed under the brand name Teflon® or nylon polymers. In some embodiments, polyacrylamide materials, for example, those marketed under the brand name Ixef® can be utilized. At least some advantages of utilizing relatively light and inexpensive materials, such as plastics and nylon polymers are that these materials are light and easy to utilize in one-handed operation by a surgeon. Furthermore, such materials are relatively inexpensive and thus ideal for construction of single-use disposable instruments.

FIG. 2 shows a front perspective view of the embodiment of FIG. 1, which provides another view of the engagement features 122. Like FIG. 1, FIG. 2 shows the forceps device 100, comprising: the body 102, the first arm 104, the second arm 106, the pivot point 108, the functional ends 110, the operational ends 112, the engagement portion 120, the engagement features 122, the opposing features 124, the first extending component 130 and the second extending component 132.

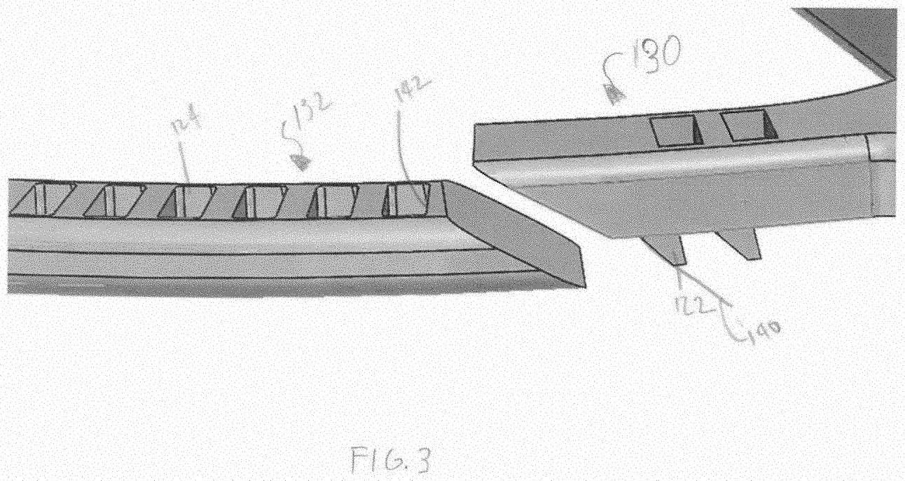

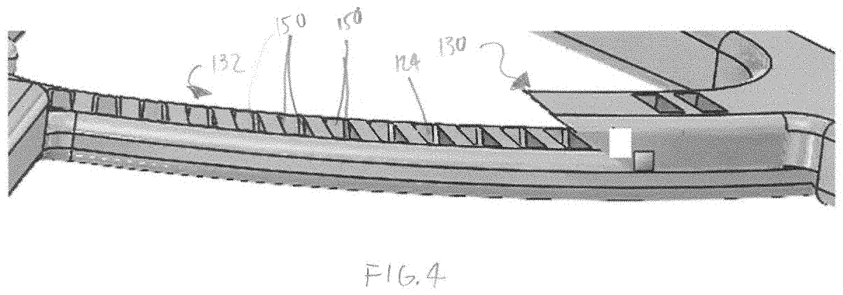

The functionality of the engagement features 122 and the opposing features 124 is also shown in the close-up operational views of FIGS. 3 and 4. FIG. 3 shows the first extending component 130 and the second extending component 132 in a disengaged configuration. FIG. 3 shows more clearly the engagement features 122, comprising protrusions in the form of teeth formed in the first extending component 130. FIG. 3 further shows more clearly the opposing features 124, comprising indentations in the form of angled holes formed in the second extending component 132. The extending components 130, 132 should have a thickness (measured from the surface having the indentations 124 to the opposite surface) which is sufficient to allow them to flex so that they can engage with one another while at the same time retaining enough rigidity to lock in place when engaged. In single-use applications the extending components may be made of plastic instead of metal and thus will require more thickness than their metal counterparts. In one embodiment, some suitable thicknesses range from 3-6 mm, although other thicknesses may suffice. The teeth 122 are angled to correspond with angled inner walls 142 of the indentations 124. In the embodiment shown, the teeth 122 are shaped to include a first backward-angled face 140, and a face of the inner wall 142 is likewise angled in a complementary fashion to correspond with the angled face 140 of the teeth 122. In one embodiment, a suitable angle would be 5-10° to ensure that the teeth 122 lock in place with the indentations 124, but it is understood that other angles are possible. In this particular embodiment, the teeth 122 protrude in a direction that is generally orthogonal to the plane in which the arms 104, 106 move during operation.

FIG. 4 shows the first extending component 130 in a position moved toward the second extending component 132, allowing the engagement features 122 to engage with the opposing features 124, such that, in this particular embodiment, the teeth (i.e., the engagement features 122) are positioned inside the holes (i.e., the opposing features 124). In this embodiment, the opposing features comprise angled holes formed within the second extending component 132 such that when the teeth of the engagement features engage the omissions, the teeth are substantially or completely surrounded on all sides by the walls 150 of the holes 124. In some embodiments, such as the embodiment shown, the walls 150 can comprise a complementary angle as discussed above with regard to FIG. 3.

Additionally, in single-use applications where the forceps 100 are made from a plastic material, for example, the extending components 130, 132 should have a suitable thickness with an acceptable degree of flexibility which allows them to flex slightly as the teeth engage but that does not flex significantly when the extending components 130, 132 are urged in the reverse direction.

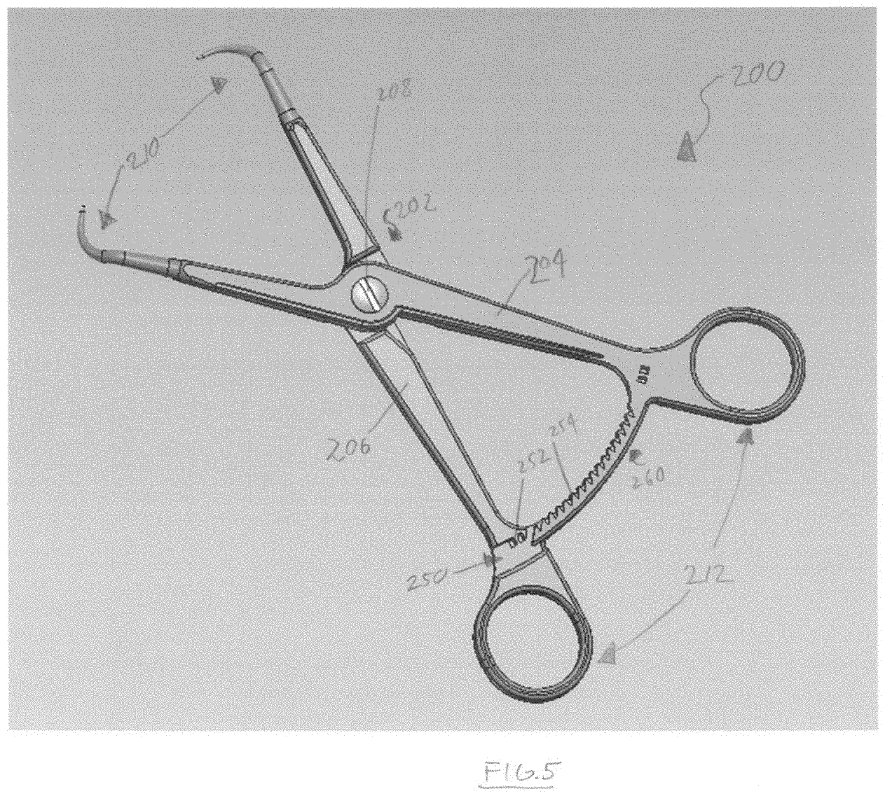

FIGS. 5-8 show another embodiment of a forceps device incorporating features of the present disclosure. FIG. shows a top perspective view of a forceps device 200, comprising: a body 202, similar to body 102 discussed above, a first arm 204, similar to the first arm 104 discussed above, a second arm 206, similar to the second arm 106 discussed above, a pivot point 208, similar to the pivot point 108 above, functional ends 210, similar to the functional ends 110 above, and operational ends 212, similar to the operational ends 112 above.

The forceps device 200 of FIG. 5 differs from the forceps device 100 of FIG. 1 in that, instead of each arm comprising extending components with complementary teeth and holes, each arm comprises a channel with opposing teeth and an extension feature with corresponding engagement teeth. The top view of FIG. 5 shows a first channel 250 formed in the second arm 206 and with opposing teeth 252 protruding into the channel 250, the opposing teeth 252 have an angled face in a complementary angle to the engagement teeth 254, formed in the first extending component 260, which is connected to the first arm 204.

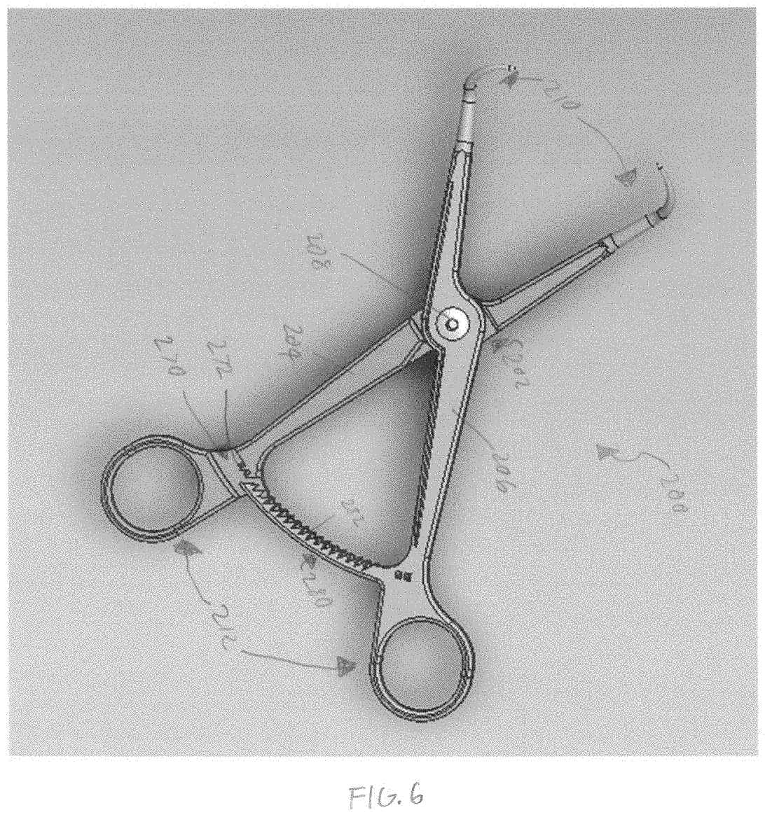

FIG. 6 shows a bottom perspective view of the forceps device 200 of FIG. 5 above, wherein the depicted reference numbers have been carried over from FIG. 5 and refer to the same features. As can be seen from this reverse view as compared to FIG. 5, the second channel 270 of the first arm 204 is shown, and it also comprises opposing teeth 272. Likewise, the second arm 206 comprises a second extending component 280, which includes engagement teeth 282. Each of the engagement teeth has a face that is disposed at an angle to complement a corresponding angled face of each of the opposing teeth 272. As can be seen from FIGS. 5 and 6, each arm 204, 206 comprises a channel 250, 270, with opposing teeth 252, 272, and each comprises an extending component 260, 280 with engagement teeth 254, 282 that are configured to engage with the opposing teeth 252, 272 as the extending component 260, 280 passes through the corresponding channel 250, 270. When engaged, the opposing teeth 252, 272 and the engagement teeth 254, 282 prevent the forceps 200 from opening and, as a result, provide a clamping force against an object, such as a bone, that is between the forceps functional ends 210.

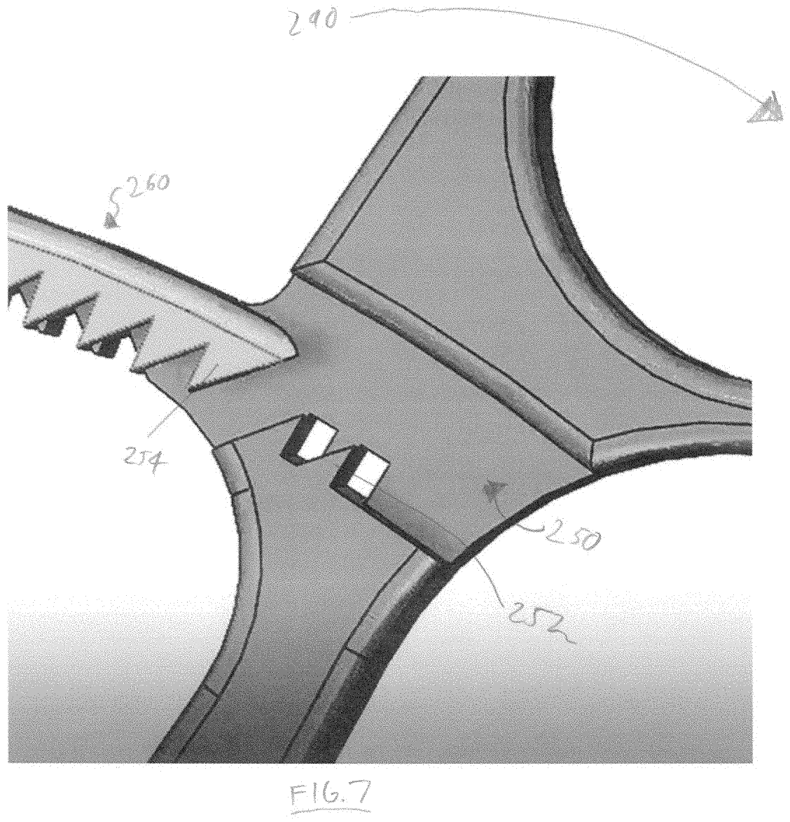

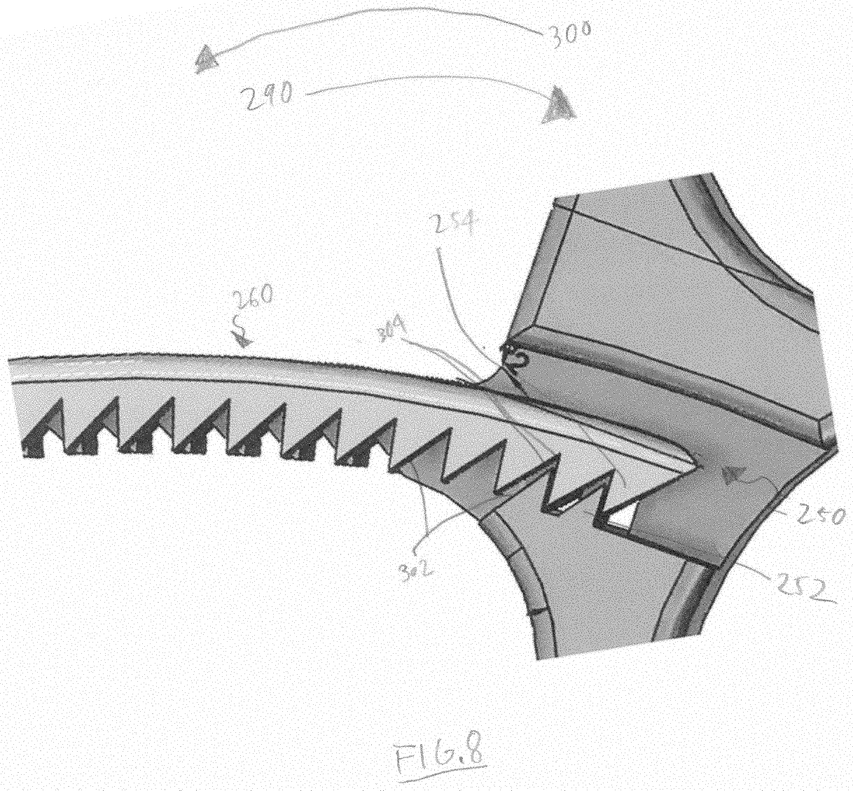

Another difference is that, unlike the embodiment of FIG. 1, the embodiment of FIG. 5 utilizes complementary opposing teeth 252, 272 rather than complementary indentations. FIGS. 7 and 8 show how the two sets of teeth engage during operation of the forceps device 200. FIG. 7 shows the first extending component 260, comprising the engagement teeth 254. FIG. 7 further shows the first channel 250, comprising the opposing teeth 252. FIG. 7 shows the engagement teeth 254 and the opposing teeth 252 in a disengaged position. However, each of the engagement teeth 254 and the opposing teeth 252 have complementary angles that allow for motion of the first extending component 260 in a forward direction 290, that is in a direction wherein the operational ends 212 of each arm 204, 206 can be moved closer together, but movement in the reverse direction is prevented. A user can only disengage the engagement teeth 254 and the opposing teeth 252 by moving the first and second arms in opposing lateral directions (i.e., in a direction that is orthogonal to the plane in which the arms 204, 206 move during operation) as with the embodiment of FIG. 1 above.

FIG. 8 shows the forceps device of FIG. 5 in an engaged position and utilizes the reference numbers of FIGS. 5-7 above. FIG. 8 shows the first extending component 260 within the first channel 250 such that the engagement teeth 254 are engaged with the opposing teeth 252. As can be seen in FIG. 8, the engagement teeth 254 and opposing teeth 252 are configured and angled such that the first extending component 260 can move in the forward direction 290, but not in the reverse direction 300, as both the engagement teeth 254 and the opposing teeth 252 comprise angled advancement portion 302, which allow the teeth 252, 254 to pass over each other, and linear stop portions 304, which block movement and prevent the teeth 252, 254 from passing over each other. The extending components 260, 280 should have a thickness (measured from the bottom of the teeth 254 to the opposite surface) which is sufficient to allow them to flex so that they can engage with the opposing teeth 252 in their respective channels 250, 270 while at the same time retaining enough rigidity to lock in place when engaged. In single-use applications, the extending components may be made of plastic instead of metal and thus will require more thickness than their metal counterparts. In one embodiment, some suitable thicknesses range from 3-6 mm, although other thicknesses may suffice.

At least one advantage of the embodiment of FIGS. 5-8 is that the configuration utilizing the channels 250, 270 which can receive the extending components 260, 280 such that the device can exhibit increased portability and handling during use. For example, the extending components 260, 280 will not need to overhang or protrude out of the channel past the arm as much. This compact configuration may improve usability by a surgeon during an operation for example.

Additionally, in single-use applications where the forceps 200 are made from a plastic material, for example, the extending components 260, 280 should have a suitable thickness with an acceptable degree of flexibility which allows them to flex slightly as the teeth engage but that do not flex significantly when the extending components are urged in the reverse direction.

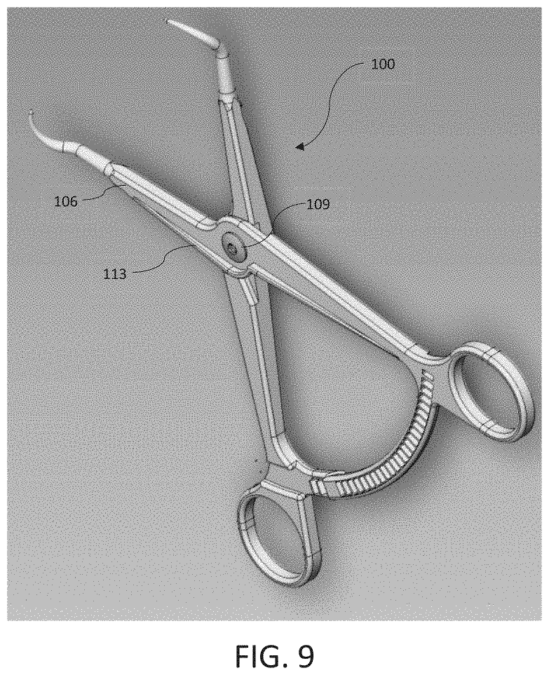

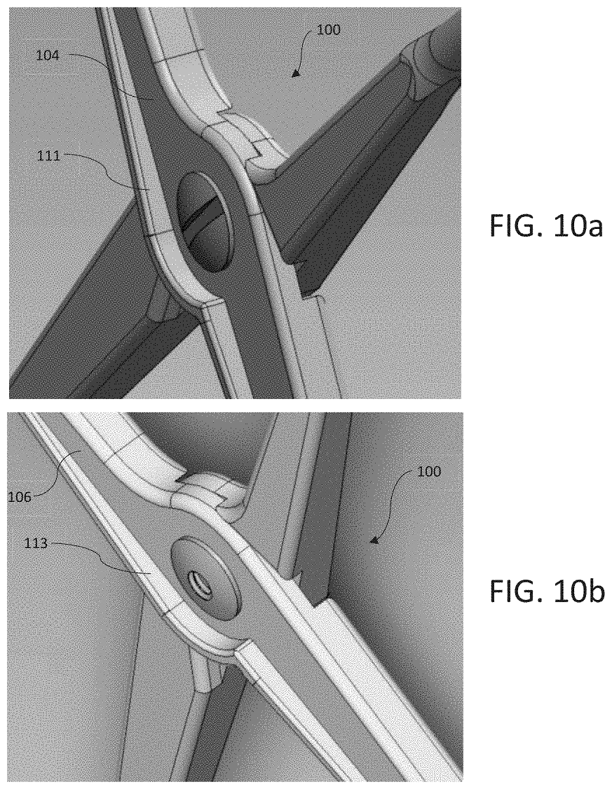

FIG. 9 is a perspective view of the forceps device 100. In this embodiment, the first and second arms 104, 106 are pivotally joined with a Chicago screw 109; although it is understood that other fasteners will also work in this capacity. Also shown in this view, is a protruding gusset or ridge 113 that runs along a perimeter of the second arm 106. A similar ridge 111 runs along the perimeter of the first arm 104. The ridge 111 is not shown in this view but is shown in FIG. 10a. The ridges 111, 113 create an I-beam type structure that provides additional rigidity and mechanical support to the arms 104, 106 when the tool is twisted to manipulate an object that the forceps 100 are holding. These ridges 111, 113 may protrude farthest from the arms 104, 106 near the pivot point 108 where the rotational stress is the greatest during operation and may then taper down to flush as the ridges 111, 113 get farther from the pivot point 108 along the arms 104, 106. The ridges 111, 113 travel around the perimeters of the arms 104, 106 near the pivot point 108 without interfering with the assembly. The arms 104, 106 are thickest in the area around the pivot point. This also provides additional mechanical strength to withstand repeated twisting of the forceps 100 during operation.

FIGS. 10a and 10b are close-up perspective views of the pivot point 108 of the forceps device 100. FIG. 10a shows the first arm 104 including ridge 111. FIG. 10b shows the second arm 106 including ridge 113. It is understood that the ridge structure can be used in forceps devices 100, 200 and in other embodiments of forceps devices not explicitly shown herein.

It is understood that while the present application is primarily directed to forceps devices utilized with medical instruments, it is understood that similar forceps or clamping devices incorporating features of the present disclosure can also be utilized in any other tool that can potentially benefit from them, for example, various tools for use with construction projects.

We claim:

1. A forceps device, comprising:
a first arm comprising a functional end and an operational end;
a second arm comprising a functional end and an operational end;
a pivot point connecting said first arm to said second arm; and
an engagement portion comprising engagement features on said first arm and complementary opposing features on said second arm, said complementary opposing features configured to engage with said engagement features.

2. The forceps device of claim 1, wherein said pivot point enables the first arm to pivot about said pivot point in relation to the second arm.

3. The forceps device of claim 1, wherein said engagement features of said first arm comprise protruding teeth and said complementary opposing features of said second arm comprise indentations.

4. The forceps device of claim 3, wherein said first arm further comprises a first extending component shaped to include said engagement features and said second arm further comprises a second extending component shaped to include said opposing features to engage with said engagement features.

5. The forceps device of claim 4, wherein said complementary opposing features comprise indentations along the entire length of said second extending component.

6. The forceps device of claim 4, wherein said engagement features comprise teeth along the entire length of said first extending component.

7. The forceps device of claim 3, wherein said protruding teeth are angled to correspond with angled inner walls of said indentations at 5° to 10° from the plane orthogonal to the plane in which said first and second arms move during operation.

8. The forceps device of claim 4, wherein said first and second extending components are configured to have a thickness of 3 mm to 6 mm.

9. The forceps device of claim 1, wherein said engagement features engage with said complementary opposing features in a direction orthogonal to a plane in which said first and second arms move during operation.

10. The forceps of device of claim 1, wherein said engagement features engage with said complementary opposing features in a direction parallel to a plane in which said first and second arms move during operation.

11. The forceps device of claim 1, wherein said device comprises Polytetrafluoroethylene (PTFE).

12. The forceps device of claim 1, wherein said device comprises polyacrylamide materials.

13. The forceps device of claim 1, wherein said first and second arms further comprise a protruding ridge running along the outer perimeter of each said arm.

14. A forceps device, comprising:
a first arm connected to a second arm at a pivot point, each of said arms comprising:
a functional end and an operational end; and
a channel with opposing teeth and an extension component with corresponding engagement teeth.

15. The forceps device of claim 14, wherein said extension components with engagement teeth in each of said arms are configured to engage with said opposing teeth in said channels in each of said arms as said extending components pass through said channels.

16. The forceps device of claim 15, where in each said channel, said engagement teeth and said opposing teeth are configured to comprise complementary angles that allow said operational ends of each arm to move closer together as the arms are closing, but prevent movement in the reverse direction is prevented unless said first and second arms are moved in opposing directions that are orthogonal to the direction said arms move to open and close.

17. The forceps device of claim 14, wherein said corresponding engagement teeth run along the entire length of said extension components.

18. The forceps device of claim 14, wherein said device is made of Polytetrafluoroethylene (PTFE).

19. The forceps device of claim 14, wherein said device is made of polyacrylamide materials.

20. The forceps device of claim 14, wherein said extension components are configured to have a thickness of 3 mm to 6 mm.

21. The forceps device of claim 15, wherein said engagement and opposing teeth have complementary but opposing angled faces, angled between 5° to 10° from the plane orthogonal to the plane in which said first and second arms move during operation.

22. The forceps device of claim 14, wherein said first and second arms further comprise a protruding ridge running along the outer perimeter of each said arm.
